(19) United States
(12) Patent Application Publication
(10) Pub. No.: US 2022/0098146 A1
(43) Pub. Date: Mar. 31, 2022

(54) REVERSIBLE IMINE UV-ABSORBERS

(71) Applicants: The United States of America, as represented by the Secretary of Agriculture, Washington, DC (US); Method Products, PBC, San Francisco, CA (US)

(72) Inventors: WILLIAM M. HART-COOPER, RICHMOND, CA (US); WILLIAM J. ORTS, BURLINGAME, CA (US); AUBRI J. THOMPSON, OAKLAND, CA (US); KAJ A. JOHNSON, SAUSALITO, CA (US)

(21) Appl. No.: 17/485,744

(22) Filed: Sep. 27, 2021

Related U.S. Application Data
(60) Provisional application No. 63/084,965, filed on Sep. 29, 2020.

(51) Int. Cl. C07C 281/18; C09B 67/42

(52) U.S. Cl. CPC C07C 281/18; C09B 67/0071

(57) ABSTRACT

Disclosed are compositions containing a UV-inhibitor, optionally a carrier, and optionally anions; wherein the UV-inhibitor is at least one compound of formula (I)

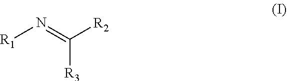

wherein R1 is an aryl derivative, N-acyl, guanidine or aminoguanidine group, and R2 and R3 are independently an aryl group substituted with a straight or branched chain alkyl or an electron donating or withdrawing group. Also disclosed are methods for protecting a product from UV light, involving applying to said product, in an amount sufficient to protect said product from UV light, the composition. Furthermore, there is disclosed methods for providing a reversible dye to a product, involving applying to said product, in an amount sufficient to provide a visible color to said product, the composition.

### REFERENCE TO RELATED APPLICATION

This application claims the benefit of U.S. Provisional Application No. 63/084,965, filed 29 Sep. 2020, which is incorporated herein by reference in its entirety.

### BACKGROUND OF THE INVENTION

Disclosed are compositions containing a UV-inhibitor, optionally a carrier, and optionally anions; wherein the UV-inhibitor is at least one compound of formula (I)

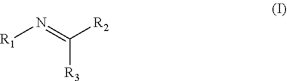

wherein R1 is an aryl derivative, N-acyl, guanidine or aminoguanidine group, and R2 and R3 are independently an aryl group substituted with a straight or branched chain alkyl or an electron donating or withdrawing group. Also disclosed are methods for protecting a product from UV light, involving applying to said product, in an amount sufficient to protect said product from UV light, a composition containing a UV-inhibitor, optionally a carrier, and optionally anions; wherein said UV-inhibitor is at least one compound of formula (I). Furthermore, there is disclosed methods for providing a reversible dye to a product, involving applying to said product, in an amount sufficient to provide a visible color to said product, a composition containing a UV-inhibitor, optionally a carrier, and optionally anions; wherein said UV-inhibitor is at least one compound of formula (I).

UV-absorbing substances protect materials from ultraviolet light, and have been used extensively in paints, coatings, adhesives, plastics, personal care products, fabrics, outdoor furniture, clothing, packaging, and textiles. UV-absorbers can be incorporated into formulas (e.g., sprays, gels, pastes) or protective coatings in order to prevent degradation of UV-sensitive materials such as polymers and colorants (Zayat, M., et al., Chemical Society Reviews, 36(8): 1270-1281 (2007)). Additionally, they are important for prevention of sunburn and long term skin damage including skin cancer (Morabito, K., et al., International Journal of Cosmetic Science, 33(5): 385-390 (2011)). Although these materials are useful and in many cases necessary, they have been associated with adverse human and environmental health attributes (Sambandan, D. R., and D. Ratner, Journal of the American Academy of Dermatology, 64(4): 748-758 (2011); Raffa, R. B., et al., Journal of Clinical Pharmacy and Therapeutics, 44(1): 134-139 (2019)). In the case of chemical UV-absorbers used as topical sunscreen ingredients, these issues include skin permeation (Janjua, N. R., et al., Journal of the European Academy of Dermatology and Venereology: JEADV, 22(4): 456-461 (2008))), skin sensitization (Funk, J. O., et al., Dermatologic Clinics, 13(2): 473-481 (1995)), endocrine disruption (Schlumpf, M., et al., Environmental Health Perspectives, 109(3): 239-244 (2001); Schlumpf, M., et al., International Journal of Andrology, 31(2): 144-151 (2008)), environmental persistence (Balmer, M. E., et al., Environmental Science & Technology, 39(4): 953-962 (2005)), aquatic toxicity (Danovaro R., et al., Environmental Health Perspectives, 116(4): 441-447 (2008); Downs, C. A., et al., Archives of Environmental Contamination and Toxicology, 70(2): 265-288 (2016); Stein, H. V., et al., Environmental Science: Processes & Impacts, 19(6): 851-860 (2017))), and sourcing from non-renewable feedstocks.

The growing concern over typical chemical UV-absorbers has created the need for new UV protection strategies. Herein we show that imines (e.g., mono- and di-hydrazine-aldehyde and amine-aldehyde hydrazones) can be effective and safe UV-absorbers.

### SUMMARY OF THE INVENTION

Disclosed are compositions containing a UV-inhibitor, optionally a carrier, and optionally anions; wherein the UV-inhibitor is at least one compound of formula (I)

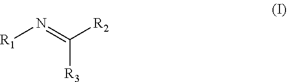

wherein R1 is an aryl derivative, N-acyl, guanidine or aminoguanidine group, and R2 and R3 are independently an aryl group substituted with a straight or branched chain alkyl or an electron donating or withdrawing group. Also disclosed are methods for protecting a product from UV light, involving applying to said product, in an amount sufficient to protect said product from UV light, a composition containing a UV-inhibitor, optionally a carrier, and optionally anions; wherein said UV-inhibitor is at least one compound of formula (I). Furthermore, there is disclosed methods for providing a reversible dye to a product, involving applying to said product, in an amount sufficient to provide a visible color to said product, a composition containing a UV-inhibitor, optionally a carrier, and optionally anions; wherein said UV-inhibitor is at least one compound of formula (I).

This summary is provided to introduce a selection of concepts in a simplified form that are further described below in the detailed description. This summary is not intended to identify key features or essential features of the claimed subject matter, nor is it intended as an aid in determining the scope of the claimed subject matter.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

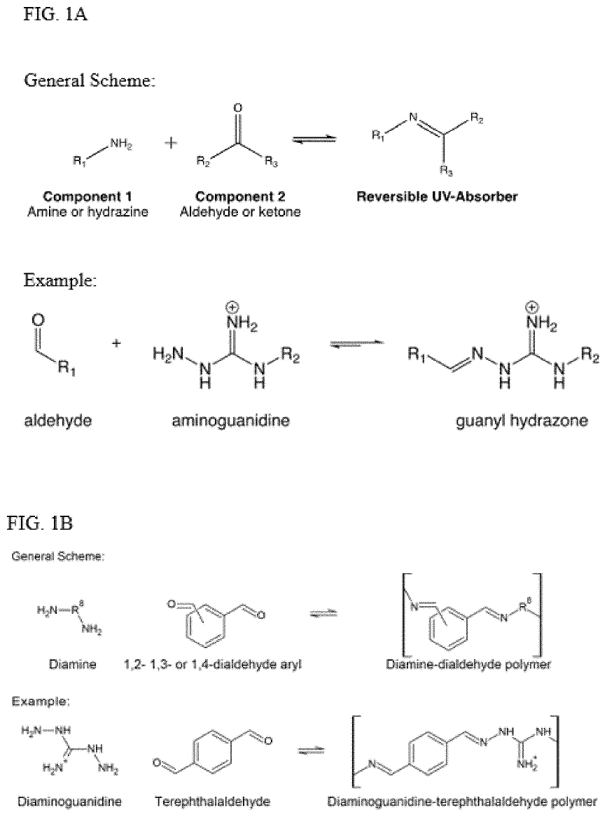

Exemplary FIG. 1A and FIG. 1B show general reaction schemes. FIG. 1A shows a general reaction schemes of reversible imine bond formation as described herein. FIG. 1B shows a general reaction scheme for reversible imine bond formation when the final compound is a polymer as described herein.

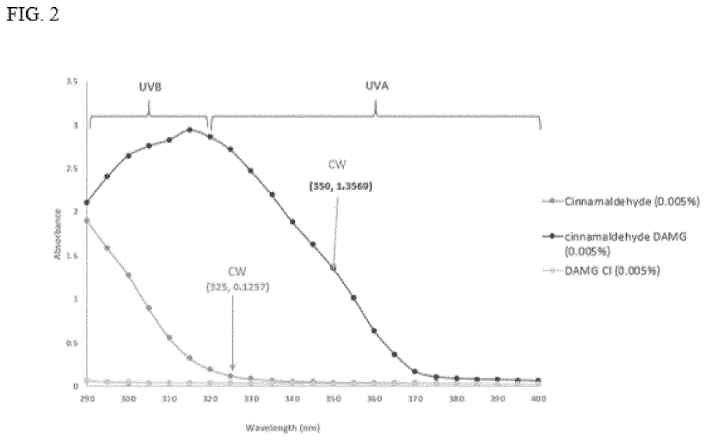

Exemplary FIG. 2 shows UV absorbance spectra of cinnamaldehyde diaminoguanidine (CinDAG) and its subcomponents aminoguanidine and aldehyde as described herein.

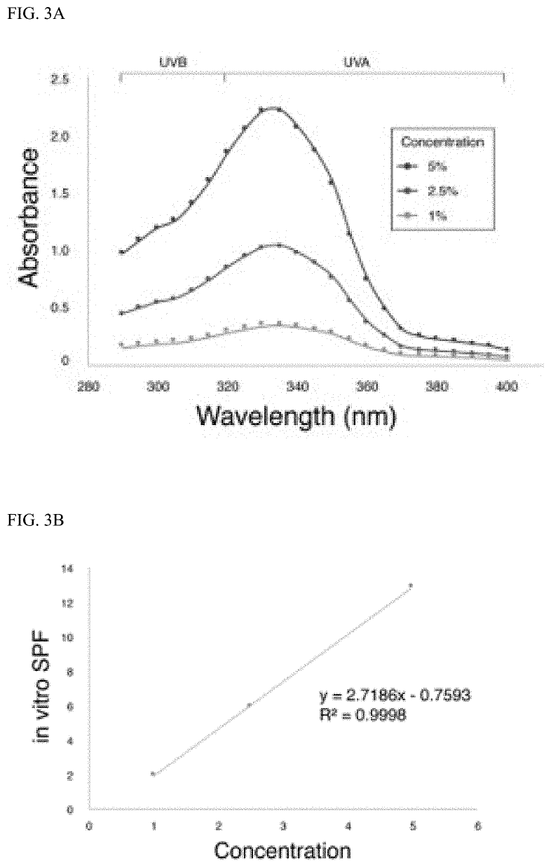

Exemplary FIG. 3A shows CinDAG UV absorbance spectrum at varying concentrations and exemplary FIG. 3B shows the corresponding graph of concentration vs. in vitro SPF (sun protection factor) with linear trendline as described herein.

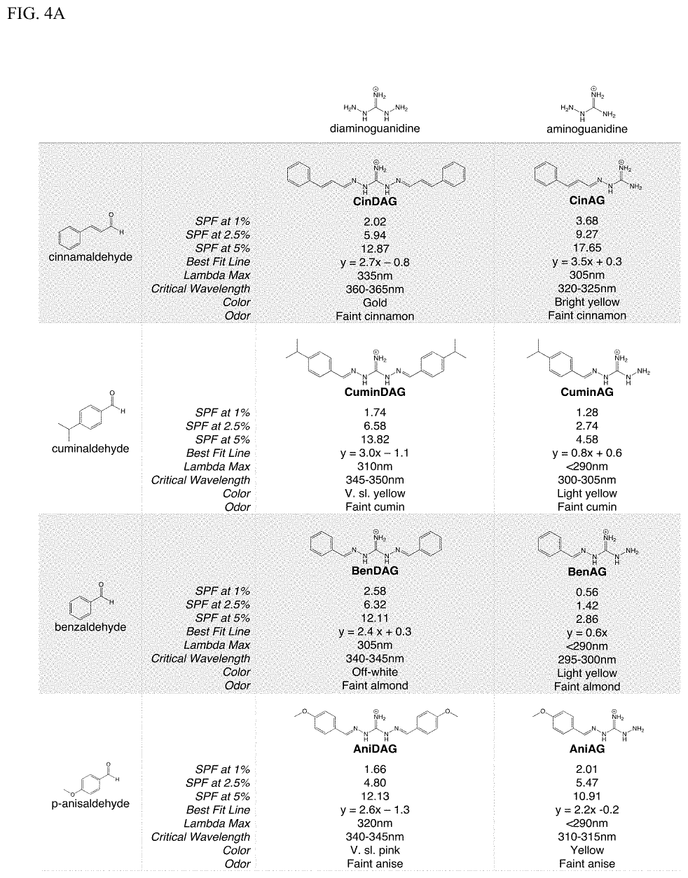

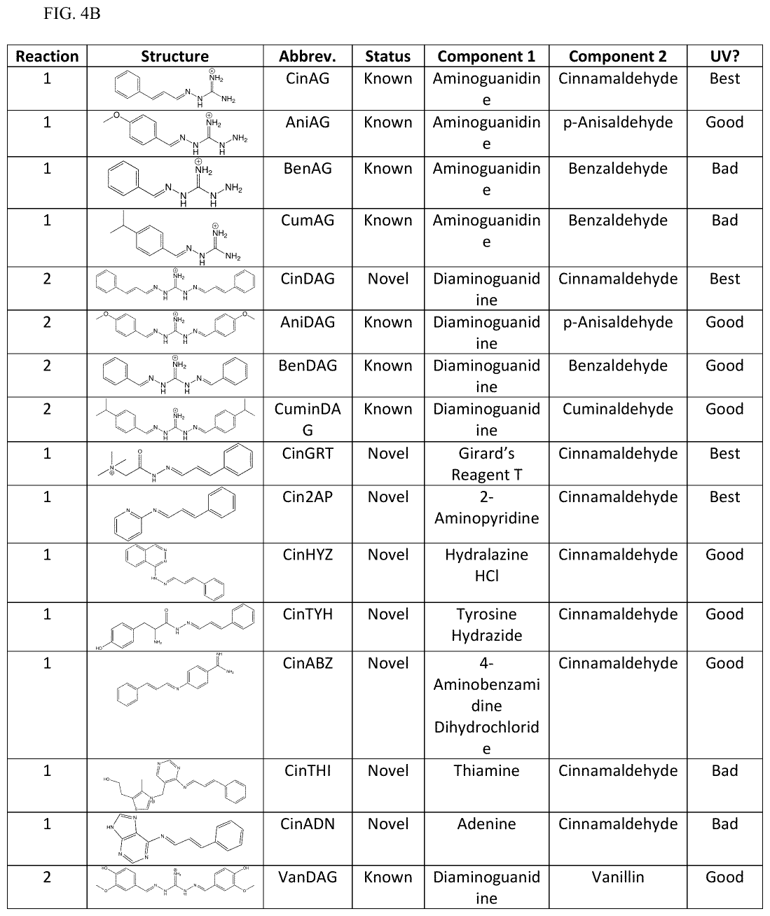

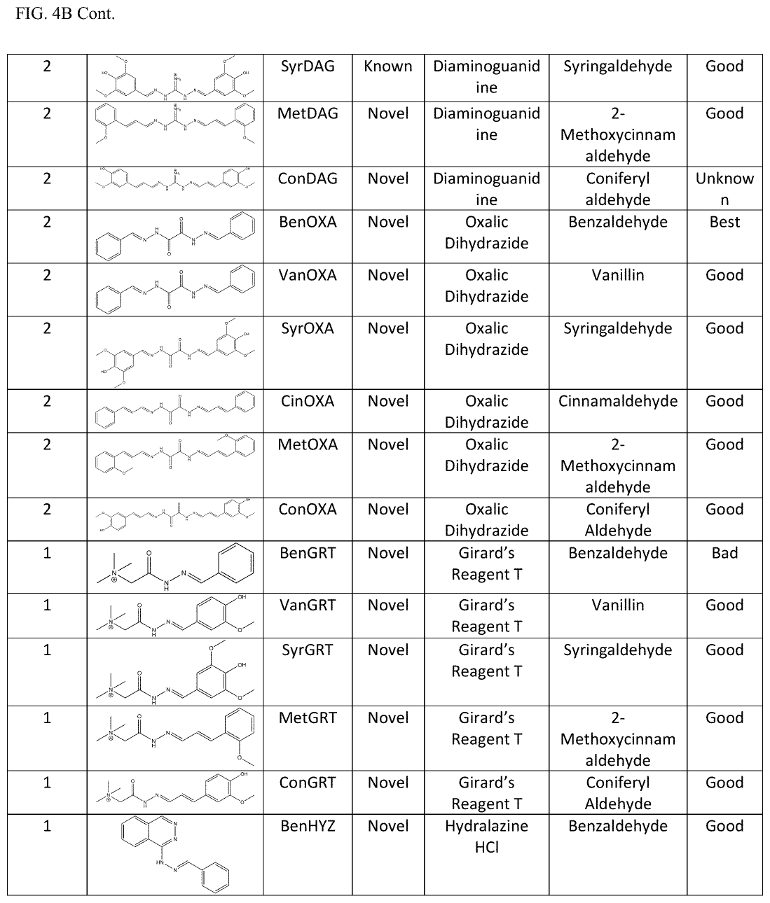

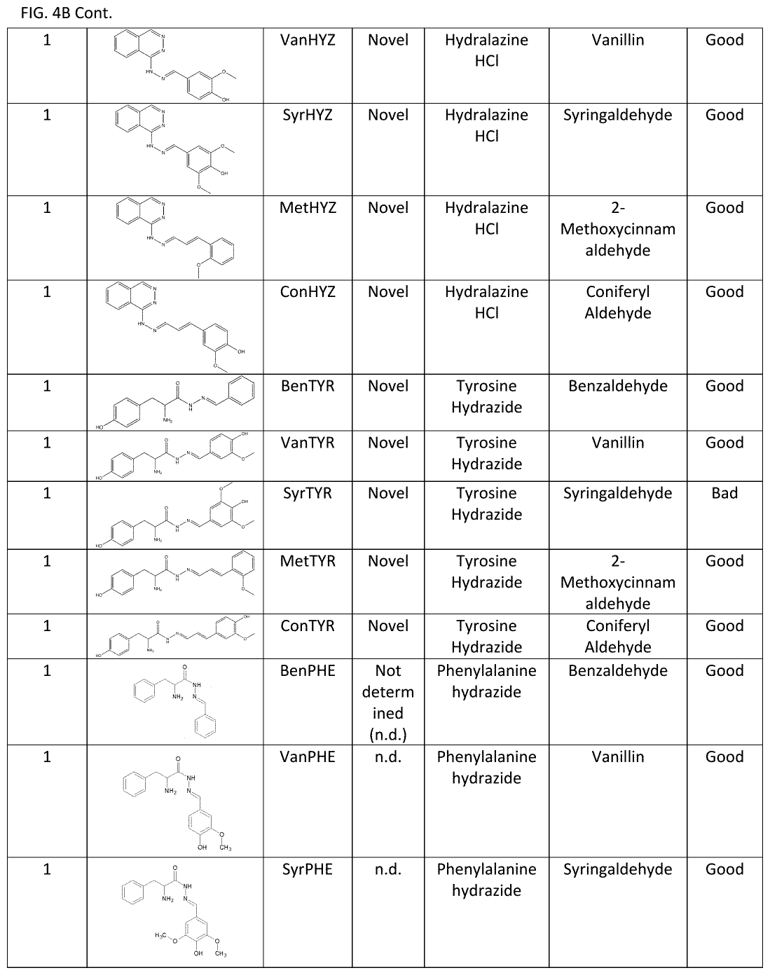

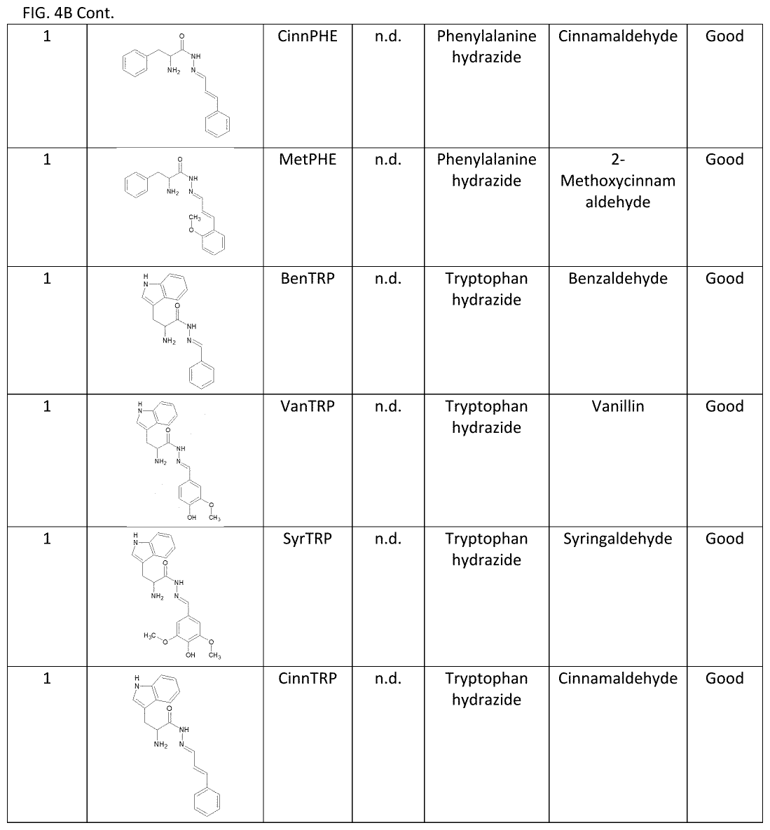

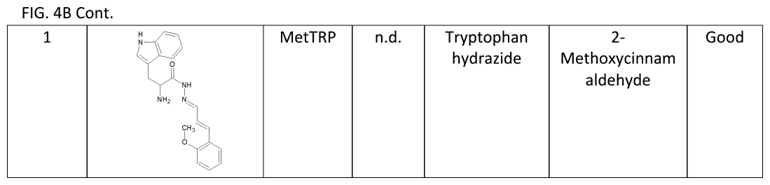

Exemplary FIG. 4A and FIG. 4B show a library of reversible sunscreens and some of their characteristics as described herein.

Exemplary FIG. 5A, FIG. 5B and FIG. 5C show comparison of cinnamaldehyde aminoguanidine (CinAG) to octinoxate as described herein; FIG. 5A shows comparison of CinAG to octinoxate through structure; FIG. 5B shows comparison of CinAG to octinoxate through UV absorbance; and FIG. 5C shows comparison of CinAG to octinoxate through in vitro SPF values.

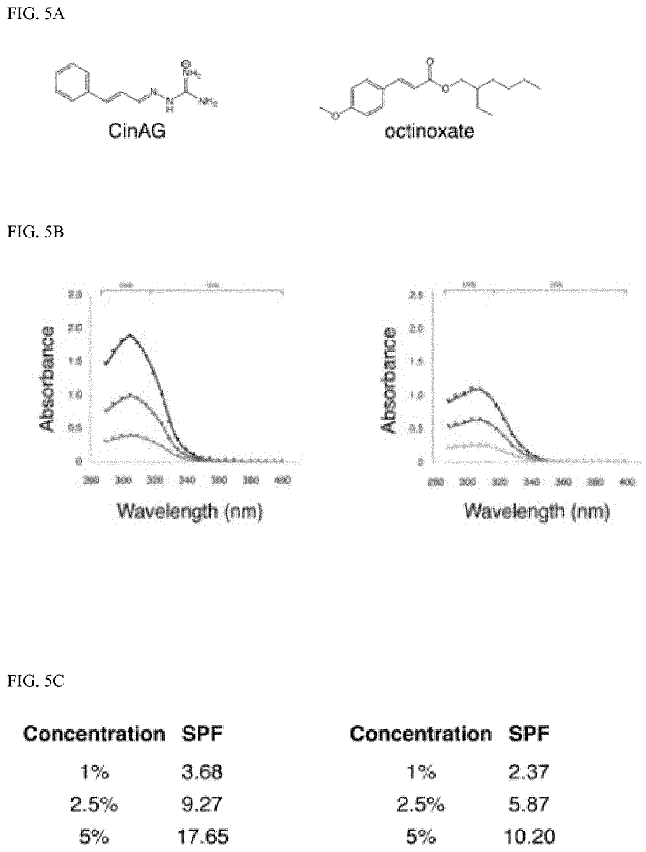

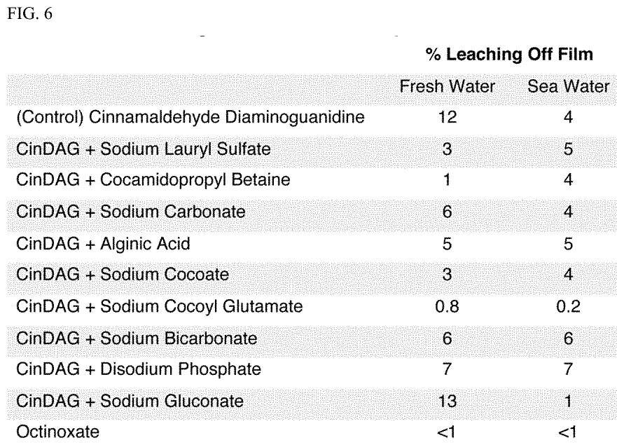

Exemplary FIG. 6 shows the effect of anion pairing on fresh water and sea water solubility as measured by percent leaching off the film as described herein.

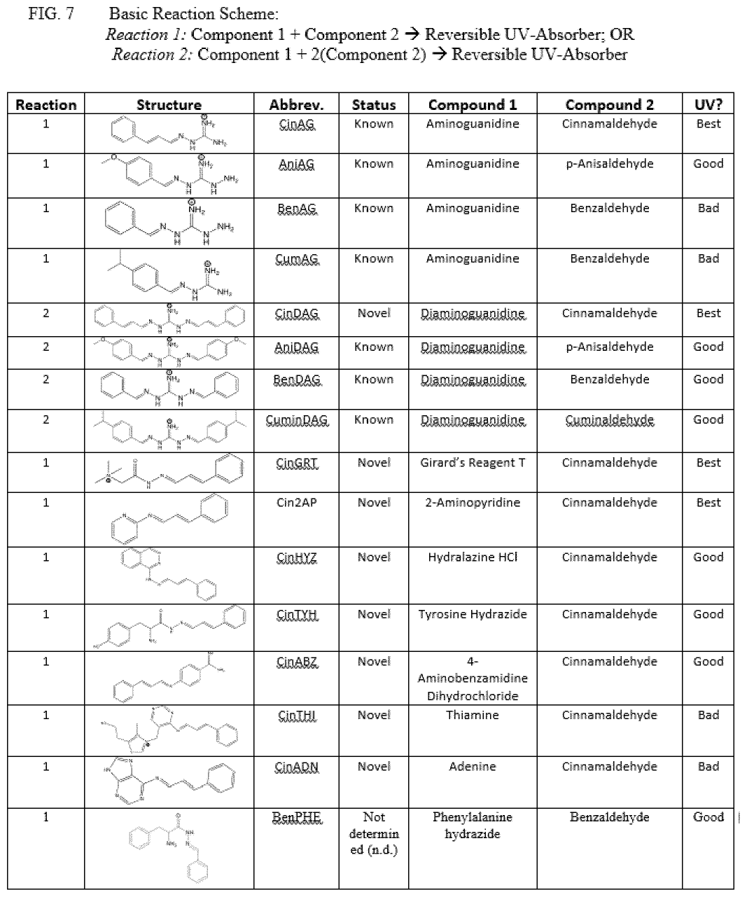

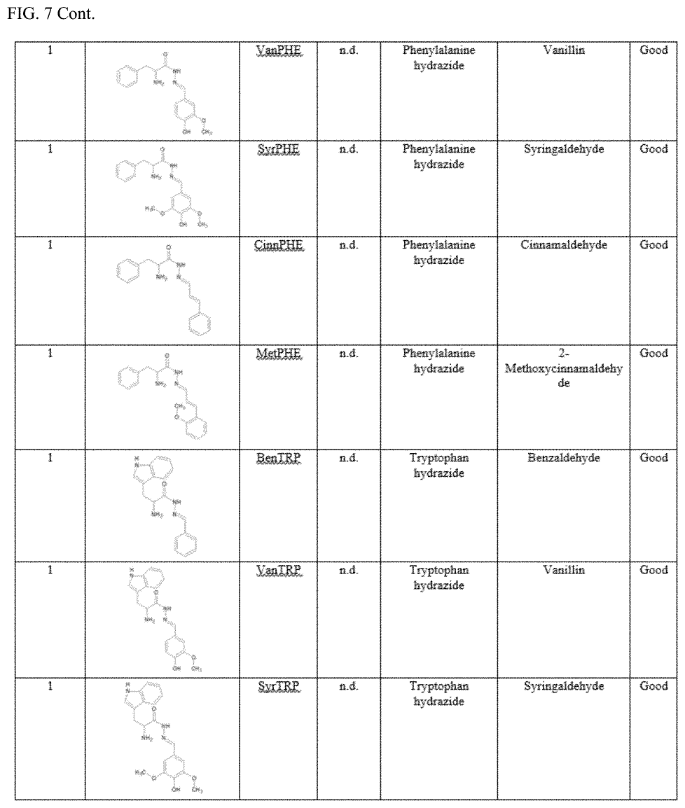

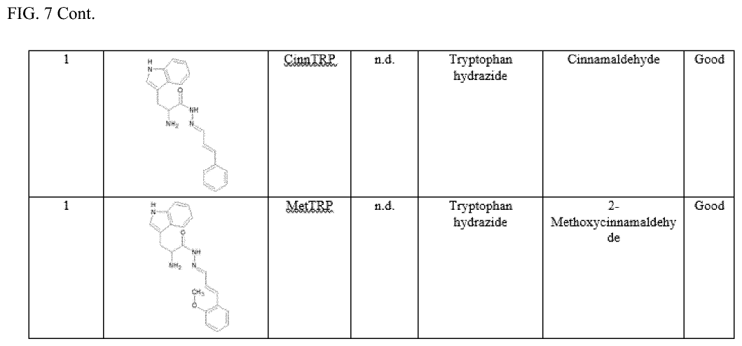

Exemplary FIG. 7 shows compounds tested as described herein.

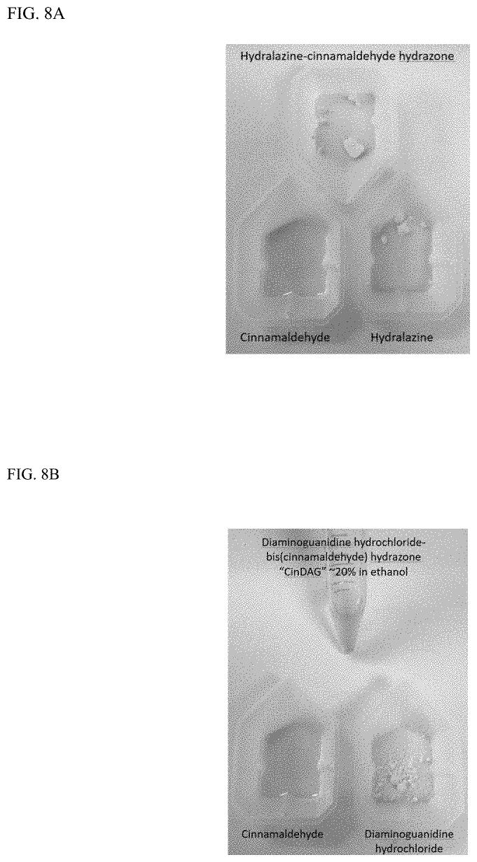

Exemplary FIG. 8A and FIG. 8B show appearance of reversible dyes in comparison to subcomponents as described herein. FIG. 8A shows cinnamaldehyde oil (light yellow) and colorless hydralazine hydrochloride afford a bright yellow hydrazine. FIG. 8B shows cinnamaldehyde oil (light yellow) and colorless diaminoguanidine hydrochloride afford a beige solid that gives a deep yellow solution when dissolved in ethanol.

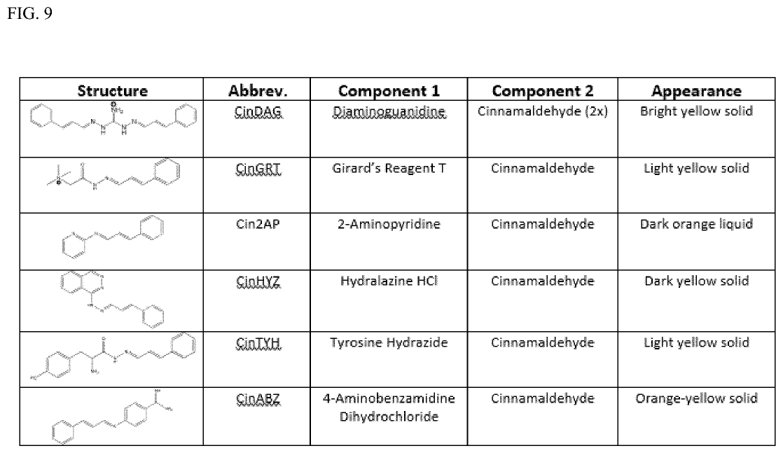

Exemplary FIG. 9 describes the appearance of certain reversible UV-absorbers that can act as dyes as described herein, including their structure, subcomponents, and color of the final dye.

### DETAILED DESCRIPTION OF THE INVENTION

Disclosed are compositions containing a UV-inhibitor, optionally a carrier, and optionally anions; wherein the UV-inhibitor is at least one compound of formula (I)

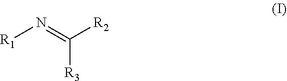

wherein R1 is an aryl derivative, N-acyl, guanidine or aminoguanidine group, and R2 and R3 are independently an aryl group substituted with a straight or branched chain alkyl or an electron donating or withdrawing group. Also disclosed are methods for protecting a product from UV light, involving applying to said product, in an amount sufficient to protect said product from UV light, a composition containing a UV-inhibitor, optionally a carrier, and optionally anions; wherein said UV-inhibitor is at least one compound of formula (I). Furthermore, there is disclosed methods for providing a reversible dye to a product, involving applying to said product, in an amount sufficient to provide a visible color to said product, a composition containing a UV-inhibitor, optionally a carrier, and optionally anions; wherein said UV-inhibitor is at least one compound of formula (I).

Herein we present a class of imines (e.g., hydrazones) designed with human and environmental health as a top priority. Repeat insult patch testing (RIPT) results indicate that aminoguanidine-aldehyde hydrazones are non-sensitizing at 1% (n=50) as shown below. From an environmental standpoint, guanyl hydrazones disassociate at high concentrations of water (Nguyen, R., and I. Huc, Chem. Commun., pages 942-943 (2003); Dirksen, et al., J. Am. Chem. Soc., 128: 15602-15603 (2006)), reducing environmental persistence. This mechanism has been widely used to create bonds that are thermodynamically stable in water (Ka˜10⁴−10⁶ M⁻¹) but kinetically reversible, with dissociation half-lives on the order of minutes to weeks (Dirksen et al. 2006; Kölmel, D. K., and E. T. Kool, Chem. Rev., 117: 10358-10376 (2017); Dirksen, A., et al., Bioconjug. Chem., 19: 2543-2548 (2008)). Some of the subcomponents we have chosen have low toxicity and are readily biodegradable (Williams, Antony J., et al., Journal of Cheminformatics, 9(1): 61 (2017)), and are sourced from inexpensive natural products such as cinnamaldehyde, cuminaldehyde, and benzaldehyde. Additionally, we can select subcomponents to form polymerized reversible UV-absorbers which have the potential to reduce skin penetration.

In addition to their positive human and environmental health attributes, we show that reversible imines (e.g., amine-aldehyde and hydrazine-aldehyde hydrazones) are effective UV-absorbers. In some cases, our reversible sunscreens outperform existing chemical UV-absorbers such as octinoxate. While aromatic aldehydes on their own have some UV absorbance, we surprisingly show that this new class of compounds has significantly higher absorbance in the desired UV range. Specifically, we examined sunscreens in the UVB range, important for preventing sunburns and correlated to sun protection factor (SPF), and the UVA range, important for preventing long term skin damage and correlated to critical wavelength (>370 nm for a ‘broad spectrum’ claim) (“Over-the-counter sunscreen drug products; required labeling based on effectiveness testing,” Electronic Code of Federal Regulations Title 21, Sec. 201.327 (21CFR201.327). Additionally, adjusting the solubility of sunscreens by pairing with an anion of low solubility product could be helpful in achieving waterproof formulations.

In addition to absorbing in the UV region, some reversible UV-absorbers can act as reversible dyes. These dyes are formed when two colorless subcomponents (e.g., one amine or hydrazine and one aldehyde or ketone) are reacted to form a colored compound. For example, cinnamaldehyde diaminoguanidine (CinDAG) is comprised of cinnmaldehyde which is a slightly yellow liquid and diaminoguanidine which is a white powder. However, the product, CinDAG, is a bright yellow powder.

Reversible dyes could be used to impart color to a product or a surface, including cleaning products and hair. They could also be used to enhance the color of an existing colored product. Because they degrade in high volumes of water, these dyes would not persist in the environment and may be a more sustainable option than petroleum based dyes. The reversible nature of the imine bond could also be used to create products that exhibit a color change when desired. For example, the subcomponents could be kept in separate packaging, and combined to create a visual effect. Additionally, the product containing an assembled dye could be diluted to cause the disappearance of color.

Exemplary FIG. 1 shows a general reaction scheme of imines (e.g., mono- and di-hydrazine-aldehyde and amine-aldehyde imines) showing reversible bond formation as described herein.

The term “product” generally includes surfaces (e.g., a hard or soft outside or uppermost part of an object, such as skin, hair, fabric, or countertop) or a formulation (e.g., mixed into a plastic film formula or mixed into a colored consumer product; any liquid or solid material incorporating the compound of formula I such as paints, coatings, adhesives, plastics, personal care items, and textiles). The term “applying” includes coating a surface (e.g., human skin, plastics, paints, coatings, printed materials, and fabrics) with the compound of formula I or mixing the compound of formula I into a formulation (e.g., paint).

The term “anion(s)” generally includes anions of low solubility that associate with the cationic compound of formula I resulting in a low solubility ion pair (in other words, resulting in an insoluble product at relevant concentrations, e.g., about 0.1 to about 10 wt %).

Other compounds (e.g., UV-absorbers known in the art) may be added to the composition provided they do not substantially interfere with the intended activity and efficacy of the composition; whether or not a compound interferes with activity and/or efficacy can be determined, for example, by the procedures utilized below.

“Optional” or “optionally” means that the subsequently described event or circumstance may or may not occur, and that the description includes instances in which said event or circumstance occurs and instances where it does not. For example, the phrase “optionally comprising a known UV-absorber” means that the composition may or may not contain a known UV-absorber and that this description includes compositions that contain and do not contain a known UV-absorber. Also, by example, the phrase “optionally adding a known UV-absorber” means that the method may or may not involve adding a known UV-absorber and that this description includes methods that involve and do not involve adding a known UV-absorber.

By the term “effective amount” of a compound or property as provided herein is meant such amount as is capable of performing the function of the compound or property for which an effective amount is expressed. As will be pointed out below, the exact amount required will vary from process to process, depending on recognized variables such as the compounds employed and the processing conditions observed. Thus, it is not possible to specify an exact “effective amount.” However, an appropriate effective amount may be determined by one of ordinary skill in the art using only routine experimentation.

The compounds described herein or compositions described herein to be used will be at least an effective amount of the compound or diluted solution of the compound. Generally the concentration of the compounds will be, but not limited to, about 0.025% to about 10% (e.g., 0.025 to 10%, for example in an aqueous solution), preferably about 0.5% to about 4% (e.g., 0.5 to 4%), more preferably about 1% to about 2% (e.g., 1 to 2%).

The compositions optionally contain a carrier (e.g., agronomically or physiologically or pharmaceutically acceptable carrier). The carrier component can be a liquid or a solid material. The term “carrier” as used herein includes carrier materials such as those described below. As is known in the art, the vehicle or carrier to be used refers to a substrate such as a mineral oil, paraffin, silicon oil, water, membrane, sachets, disks, rope, vials, tubes, septa, resin, hollow fiber, microcapsule, cigarette filter, gel, fiber, natural and/or synthetic polymers, elastomers or the like. All of these substrates have been used to controlled release effective amount of a composition containing the compounds disclosed herein in general and are well known in the art. Suitable carriers are well-known in the art and are selected in accordance with the ultimate application of interest. Agronomically acceptable substances include aqueous solutions, glycols, alcohols, ketones, esters, hydrocarbons halogenated hydrocarbons, polyvinyl chloride; in addition, solid carriers such as clays, laminates, cellulosic and rubber matrices and synthetic polymer matrices, or the like.

While this invention may be embodied in many different forms, there are described in detail herein specific preferred embodiments of the invention. The present disclosure is an exemplification of the principles of the invention and is not intended to limit the invention to the particular embodiments illustrated. All patents, patent applications, scientific papers, and any other referenced materials mentioned herein are incorporated by reference in their entirety. Furthermore, the invention encompasses any possible combination of some or all of the various embodiments and characteristics described herein and/or incorporated herein. In addition, the invention encompasses any possible combination that also specifically excludes any one or some of the various embodiments and characteristics described herein and/or incorporated herein.

The amounts, percentages and ranges disclosed herein are not meant to be limiting, and increments between the recited amounts, percentages and ranges are specifically envisioned as part of the invention. All ranges and parameters disclosed herein are understood to encompass any and all subranges subsumed therein, and every number between the endpoints. For example, a stated range of “1 to 10” should be considered to include any and all subranges between (and inclusive of) the minimum value of 1 and the maximum value of 10 including all integer values and decimal values; that is, all subranges beginning with a minimum value of 1 or more, (e.g., 1 to 6.1), and ending with a maximum value of 10 or less, (e.g. 2.3 to 9.4, 3 to 8, 4 to 7), and finally to each number 1, 2, 3, 4, 5, 6, 7, 8, 9, and 10 contained within the range.

Unless otherwise indicated, all numbers expressing quantities of ingredients, properties such as molecular weight, reaction conditions (e.g., reaction time, temperature), percentages and so forth as used in the specification and claims are to be understood as being modified in all instances by the term “about.” Accordingly, unless otherwise indicated, the numerical properties set forth in the following specification and claims are approximations that may vary depending on the desired properties sought to be obtained in embodiments of the present invention. As used herein, the term “about” refers to a quantity, level, value, or amount that varies by as much as 10% to a reference quantity, level, value, or amount. For example, about 1.0 g means 0.9 g to 1.1 g and all values within that range, whether specifically stated or not.

Unless defined otherwise, all technical and scientific terms used herein have the same meaning as commonly understood by one of ordinary skill in the art to which the invention belongs. The definitions herein described may or may not be used in capitalized as well as singular or plural form herein and are intended to be used as a guide for one of ordinary skill in the art to make and use the invention and are not intended to limit the scope of the claimed invention. Mention of trade names or commercial products herein is solely for the purpose of providing specific information or examples and does not imply recommendation or endorsement of such products. Although any methods and materials similar or equivalent to those described herein can be used in the practice or testing of the present invention, the preferred methods and materials are now described.

The following examples are intended only to further illustrate the invention and are not intended to limit the scope of the invention as defined by the claims.

### EXAMPLES

General procedure for synthesizing reversible UV-absorbers aminoguanidine-aldehyde imines. These compounds were reversibly assembled through reaction of Component 1 (e.g., an amine or hydrazine) aminoguanidine chloride and Component 2 aldehyde in the correct molar ratios (1:2 for dihydrazines:aldehydes, 1:1 for mono-amines and hydrazines:aldehydes). In some cases, we chose to use dialdehydes to create larger, polymerized UV-absorbers. These require a molar ratio of 1:1 for dihydrazines:dialdehydes and 2:1 for mono-amines and hydrazines:dialdehydes. The Component 1 (e.g., an amine or hydrazine) aminoguanidine chloride was solubilized in water and the aldehyde was separately solubilized in ethanol. The two solutions were combined under stirring, and the mixture was brought to 55° C. for 30 minutes. Additional ethanol was added as necessary during this time to solubilize the product. The resulting mixture was left in the hood overnight to allow for ethanol evaporation. The product was examined organoleptically and confirmed by ¹H NMR and ¹³C NMR (Messeder, J. C., et al., Bioorganic & Medicinal Chemistry Letters, 5(24): 3079-3084 (1995)).

General procedure for determining in vitro SPF and critical wavelength: This method was adapted from Dutra, et al. (Dutra, E. A., et al., Brazilian Journal of Pharmaceutical Sciences, 3: 381-385 (2004)). First, 1.0 gram of sunscreen was placed in 100 mL flask and filled to volume with ethanol. The solution was mixed and filtered through cotton. 1.0 mL of the filtered solution was added to a 50 mL volumetric flask and the flask was filled to volume with ethanol. This final solution (˜0.02% of initial sunscreen) was placed in a 1 cm polystyrene cuvette and UV/Vis spectrum was recorded from 290 to 400 nm, every 5 nm, using a UV/Vis spectrophotometer (Molecular Devices Spectramax M2 UV-Vis Spectrophotometer) with ethanol as a reference . Next, the Mansur equation was applied (Mansur, J. S., et al., An Bras Dermatol., 61: 121-124 (1986)):

$$SPF = CF \times {\sum }_{290}^{320} EE ( λ ) \times I ( λ ) \times abs ( λ ) \qquad ( 1 )$$

where EE (λ)=erythemal effect spectrum; I(λ)=solar intensity spectrum; abs (λ)=absorbance of sunscreen product; CF—correction factor (=10). The values of EE×I are constants determined by Sayre et al. (Photochemistry and Photobiology, 29(3): 559-566 (1979)) so that a standard sunscreen formulation containing 8% homosalate presents an SPF value of 4. The critical wavelength was determined as the wavelength at which 90% of the total absorbance from 290-400 nm occurred. Error can be attributed to volumetric dilutions and error intrinsic to a UV/Vis spectrophotometer.

General protocol to assess film leaching: To a polyethylene square (1 cm by 1 cm, cut from a Ziploc® bag) 10 μL of a concentrated solution (˜5 wt % in EtOH) of imine was applied and allowed to dry at room temperature (10-20 min) before a concentrated solution of the anion (e.g., sodium lauryl sulfate, sodium cocoyl glutamate, sodium carbonate) to be tested (˜5 wt %) was co-applied and again allowed to dry on the film surface. The film sample was placed inside a 1.5 mL plastic tube containing 1 mL of DI or sea water (sourced from San Francisco Bay, Albany, Calif.) and after a set time (10 min), analyzed for the presence of soluble imine, which was quantified as a percentage of total sample that was originally present on the film (FIG. 6).

We determined the UV absorbing capacity of guanyl hydrazones in comparison to their aminoguanidine and aldehyde subcomponents. We prepared cinnamaldehyde diaminoguanidine (CinDAG) by reacting its subcomponents, diaminoguanidine and cinnamaldehyde, in a molar ratio of 1:2. UV spectroscopy was performed and it was surprisingly determined that the guanyl hydrazone had significantly more UV-absorbing capacity than its subcomponents, and that this absorption occurred in the UVB and UVA regions which are important for SPF and broad spectrum considerations.

In order to catalog the effects of subcomponent selection, we prepared a library of reversible sunscreens by reacting an aldehyde (e.g., cinnamaldehyde, cuminaldehyde, benzaldehyde, and anisaldehyde) with an aminoguanidine (e.g., aminoguanidine and diaminoguanidine). Products were confirmed organoleptically and by NMR. The molecules were solubilized in propanediol to form sunscreens of varying concentrations (1%, 2.5%, 5%), and in vitro SPF was determined through UV spectroscopy and application of the Mansur equation (Mansur et al. 1986). This allowed for the calculation of a best fit line and the confirmation that sunscreen concentration is related linearly to the in vitro SPF as shown in FIG. 3.

Additionally, the lambda max and critical wavelength were determined for the library of sunscreens. Organoleptic observations were also performed, and the results are summarized in FIG. 4A and FIG. 4B. The library approach showcases the flexibility of our technology to be optimized for certain characteristics. For example, if a broad-spectrum sunscreen is desired, CinDAG may be selected due to its high critical wavelength; whereas CinAG would be the best choice for solely SPF considerations.

In order to evaluate comparative efficacy, these results were compared to the commercial chemical sunscreen, octinoxate. Octinoxate is approved by the FDA for use as a sunscreen active up to 7.5% (Klein, K., Cosmetics Toiletries, 107: 45-63 (1992)), although it has been banned in Hawaii for causing damage to coral reefs (Zayat et al. 2007). Octinoxate sunscreens were prepared at various concentrations in ethanol due to insolubility in propanediol. We surprisingly found that several reversible sunscreens in our library achieved similar SPF values to octinoxate, with CinAG surprisingly outperforming octinoxate by 155% (at a concentration of 1% in formula).

As an additional feature, reversible UV-absorbers (e.g., aminoguanidine-aldehyde condensation products) can be paired with anions (e.g., that associate with the cation at typical use levels, e.g., 0.1-10 wt %) to manipulate solubility characteristics. This can greatly reduce water solubility to produce a waterproof sunscreen without relying on increased hydrophobicity of the active agent which is related to increased skin penetration (Wilschut, A., et al., Chemosphere, 30: 1275-1296 (1995); Guy, R. H., and R. O. Potts. American Journal of Industrial Medicine, 23(5): 711-719 (1993))). Results are summarized in FIG. 6. Adding sodium cocoyl glutamate to CinDAG surprisingly decreased solubility in both fresh and salt water, making it an ideal combination for a waterproof sunscreen. The water resistance of the blend is comparable to that of octinoxate, with predicted reduced skin permeation due to lower hydrophobicity.

S1: Calculation of Skin Permeation using Modified Potts and Guy Model: The Modified Potts and Guy Model is a validated method for calculating the skin permeation coefficient (Kₚ) of a compound in cm/hr (Wilschut et al. 1995; Guy and Potts 1993). It relies on two inputs—the octanol-water partition coefficient, (K_{ow}, indicative of hydrophobicity), and the molecular weight of the compound as follows:

$$log ( {K}_{p} ) = 0.688 log ( {K}_{ow} ) - 0.181 \sqrt{( MW )} - 1.525 \qquad ( S1 )$$

Using the Modified Potts and Guy Model, we calculated the skin permeation coefficient (Kₚ) of aminoguanidine and aldehyde subcomponents. This scenario represents the skin permeation of our compounds after dissociation. In order to probe skin permeation of the assembled guanyl hydrazones, which don't have published K_{ow}'s, we calculated Kₚ's of structurally similar molecules, robenidine and guanabenz. This scenario represents the application of our UV-absorbers directly to the skin via topical products. Lastly, we calculated the Kₚ of octinoxate for comparison. These values are shown in Table 1. Based on these theoretical values, our compounds should have a much lower skin permeation potential than octinoxate and other commercial UV-absorbers.

S2: Repeat Insult Patch Testing (RIPT) Procedure and Results: Human repeat insult patch testing for skin irritation and skin sensitization evaluations, including standard patient consent procedures and oversight, which were obtained after the nature and possible consequences of the study were explained, were conducted by BioScreen Testing Services, Inc. (Torrance, Calif.). Fifty-two volunteers were recruited, provided informed consent prior to initiating, and completed the study (ages 20-59, 10 male, 42 female, all with Fitzpatrick Skin Type 3—burn moderately, tan progressively). Approximately 0.02-0.05 mL of commercial formula (Daily Shower spray cleaner containing 0.2 or 1.0% cuminaldehyde aminoguanidine (CuminAG), pH 5.5-5.9; diluted 3% in DI water) was dispensed on a 7.5 mm paper disk or Rayon/polypropylene patch, which were then affixed to the skin of the intrascapular regions of the back. Subjects were instructed not to wet or expose the test area to sunlight. Patches were kept in place for 48 h after the first application, and 24 h after subsequent applications. This procedure was repeated until nine consecutive 24 h exposures had been made three times a week for three consecutive weeks. Test sites were evaluated by a trained laboratory personnel before each reapplication. After a 10-14 day rest period, a challenge dose was applied once to a previously unexposed test site, which was evaluated by a trained personnel after 48 and 96 h. The International Contact Dermatitis Research Group scoring scale was used: 0=no reaction, 1=erythema throughout at least ¾ of patch area, 2=erythema and induration throughout at least ¾ of patch area, 3=erythema, induration and vesicles, 4=erythema, induration and bullae. Surprisingly no adverse reactions of any kind were reported during the study using the Daily Shower formula containing CuminAG. Four subjects exhibited a Grade 1 reaction and two subjects a Grade 2 reaction to the positive control (2.0% sodium lauryl sulfate solution). No subjects showed any signs of reaction to the negative control (DI water).

S3: Toxicity and Biodegradation of Subcomponents: In order to evaluate aquatic toxicity and biodegradation of our compounds, we used The CompTox Chemistry Dashboard from the United States Environmental Protection Agency (Williams, A. J., et al., The CompTox Chemistry Dashboard: a community data resource for environmental chemistry, J Cheminform 9: 61 (2017), https://doi.org/10.1186/s13321-017-0247-6). We performed analysis of aminoguanidine and aldehyde subcomponents as we assumed the dissociation of guanyl hydrazones at dilute concentrations. We used the U.S. EPA guidelines for categorizing hazard (“Methodology for risk-based prioritization under ChAMP,” Office of Pollution Prevention and Toxics, U.S. Environmental Protection Agency): (1) For acute oral toxicity: high hazard=LD50<50, moderate hazard=LD50 50-500, and low hazard=LD50>500. (2) For acute aquatic toxicity: high hazard=LC50<1, moderate hazard=LC50 1-10, and low hazard=LC50>10. (3) For biodegradation: rapid=half-life<2 days, moderate=half-life 2 days-2 months, slow to negligible=half-life>2 months. The results are compiled and color-coded in Table 2. These results support the surprising low toxicity and fast biodegradation of aminoguanidine-aldehyde UV-absorbers.

Conclusions: In this investigation, we propose the use of aminoguanidine-aldehyde hydrazones and related imine derivatives as reversible sunscreens. These molecules are designed to decrease the negative effects of traditional UV-absorbers such as skin penetration, endocrine disruption, skin sensitization, environmental persistence, aquatic toxicity, and non-renewable sourcing. In addition to improved human and environmental health attributes, these reversible sunscreens perform similarly to the commercial UV-absorber octinoxate. Cinnamaldehyde aminoguanidine (CinAG) was surprisingly the highest performing from an SPF perspective with a 155% SPF improvement over octinoxate at 1% in formula, and different subcomponents can be selected to optimize critical wavelength or organoleptic characteristics. Additionally, dialdehydes can be used to create polymerized reversible UV-absorbers (FIG. 1B). Lastly, by pairing reversible sunscreens with an anion of low solubility product, water solubility can be reduced. Specifically, pairing cinnamaldehyde diaminoguanidine (CinDAG) with sodium cocoyl glutamate is ideal for a waterproof sunscreen product.

By introducing a new class of bio-based, low toxicity UV-absorbers, we aim to offer an alternative in light of growing concerns over current chemical sunscreens. Our solution offers similar or improved efficacy with vastly improved human and environmental health impacts. These attributes make our reversible UV-absorbers useful for an array of UV protective products including topical sunscreens, color cosmetics, plastics, paints, coatings, printed materials, and fabrics.

Due to the colored nature of many reversible UV-absorbers, we also foresee their use as degradable dyes and color enhancers that could minimize the sourcing concern and aquatic toxicity of petroleum based dyes. In addition, reversible dyes could be used to create novel color change effects. This technology would have applications in a range of products including color cosmetics, cleaning products, laundry care, paints, coatings, printed materials, and fabrics.

All of the references cited herein, including U.S. Patents and U.S. Patent Application Publications, are incorporated by reference in their entirety, including the following: U.S. Pat. Nos. 7,815,900; 10,227,501; Li, Jian, et al., Toxicology in Vitro, 24(1): 201-207 (2010).

Thus, in view of the above, there is described (in part) the following:

A method for protecting a product from UV light (e.g., blocks UVA from about 100 to about 400 nm, preferably about 290 to about 400 nm), said method comprising (or consisting of or consisting of) applying to said product, in an amount sufficient to protect said product from UV light, a composition comprising (or consisting of or consisting of) a UV-inhibitor, optionally a carrier, and optionally anions; wherein said UV-inhibitor is at least one compound of formula (I)

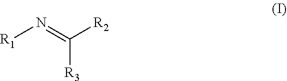

wherein R1 is an aryl derivative (is a planar aromatic compound substituted with simple R groups such as —H, alkyl groups, —OH, —OCH₃, —NH₂; for example 4-aminobenzamidine dihydrochloride)), hydralazine, pyridine, N-acyl (e.g., Girard's reagent T and Tyrosine hydrazide), guanidine or aminoguanidine group, and R2 and R3 are independently hydrogen or an aryl group substituted with a straight or branched chain alkyl (e.g., C1-10, preferably C1-6, more preferably C1-4 (e.g. —CH₃, —CH₂CH₃, —CH(CH₃)₂, —CH₂CH₂CH₃)) or an electron donating or withdrawing group (e.g., —COOH, —OH, —OCH₃, —OCH₂CH₃, —NH₂, —NHCH₃, —N(CH₃)₂, —NO₂, —Cl, —Br, —F, -Ph; specific examples include Ph-CH₃ and Ph-Cl). Electron withdrawing group (EWG): A functional group that draws electron density from neighboring atoms towards itself, usually by resonance or inductive effects; examples include —NR3+, —F/Cl/Br/I (halogens can be donating through their pi system and withdrawing by induction), —CO₂R, —NO₂). Electron donating group is a group with lone pairs available for pi bonding (e.g., halogens, carboxyls, aminos, etc).

The above method, wherein R1 is

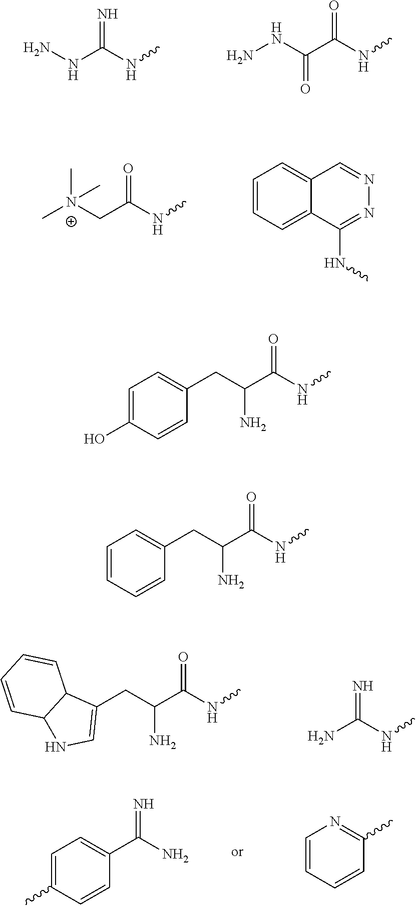

and wherein R2 and R3 are (R2 or R3 would have to be —H for any aldehyde subcomponent)

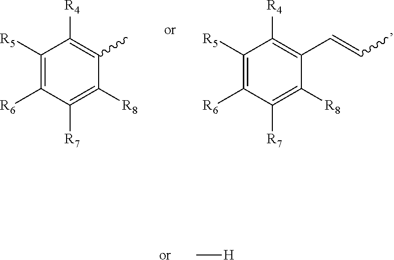

wherein R4 through R8 are independently hydrogen, a straight or branched chain alkyl (e.g., C1-10, preferably C1-6, more preferably C1-4 (e.g., —CH₃, —CH₂CH₃, —CH(CH₃)₂, —CH₂CH₂CH₃)) or an electron donating or withdrawing group (e.g., —COOH, —OH, —OCH₃, —OCH₂CH₃, —NH₂, —NHCH₃, —N(CH₃)₂, —NO₂, —Cl, —Br, —F, -Ph).

The above method, wherein said UV-inhibitor is at least one of the following compounds:

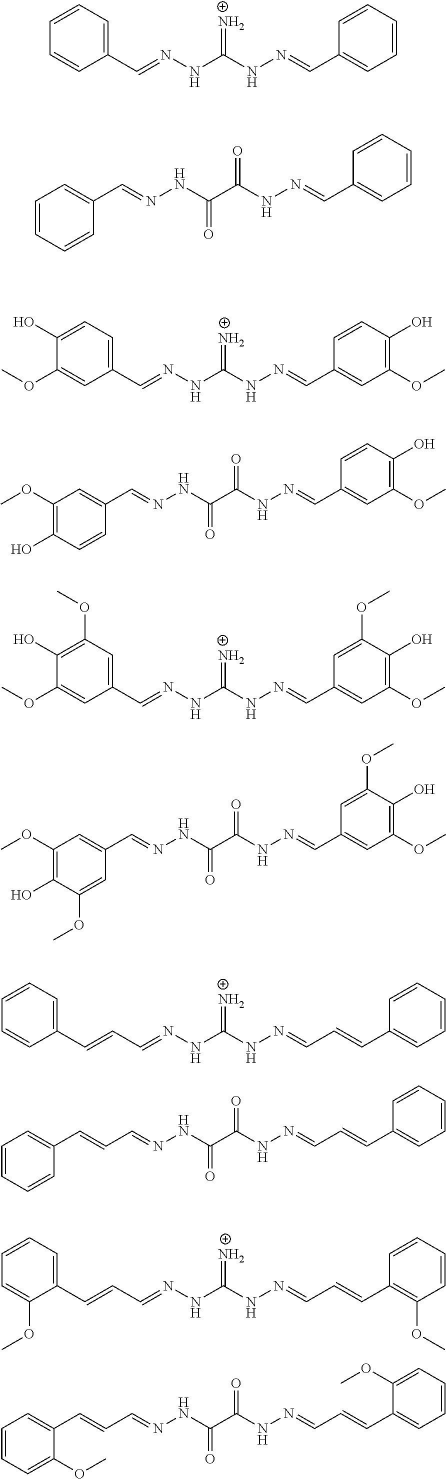

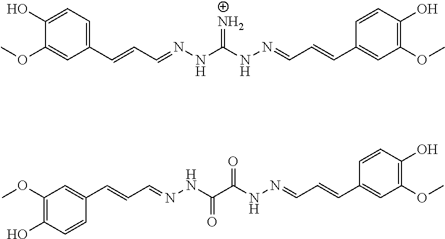

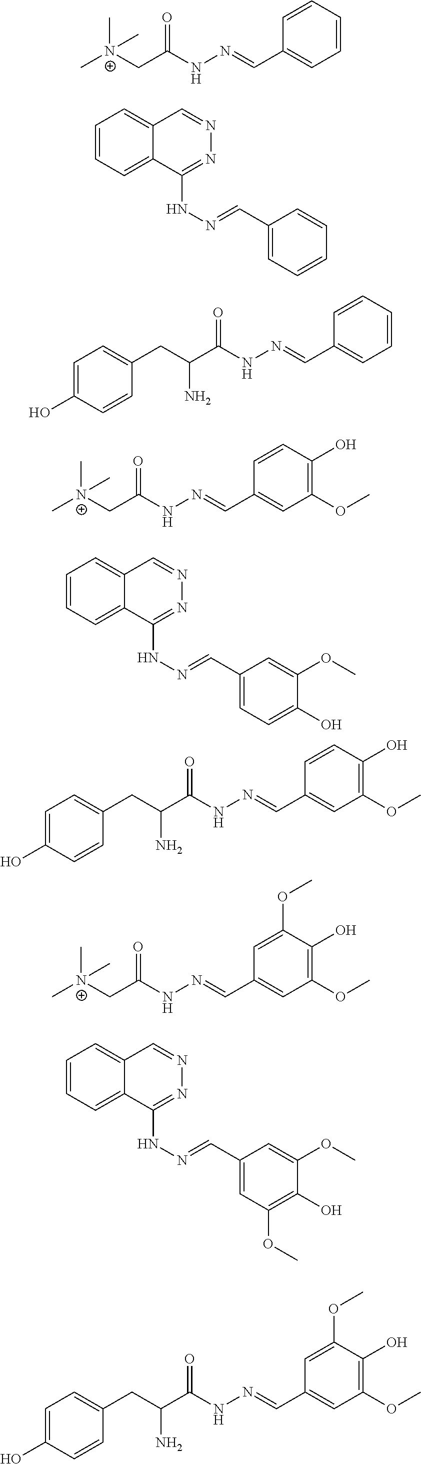

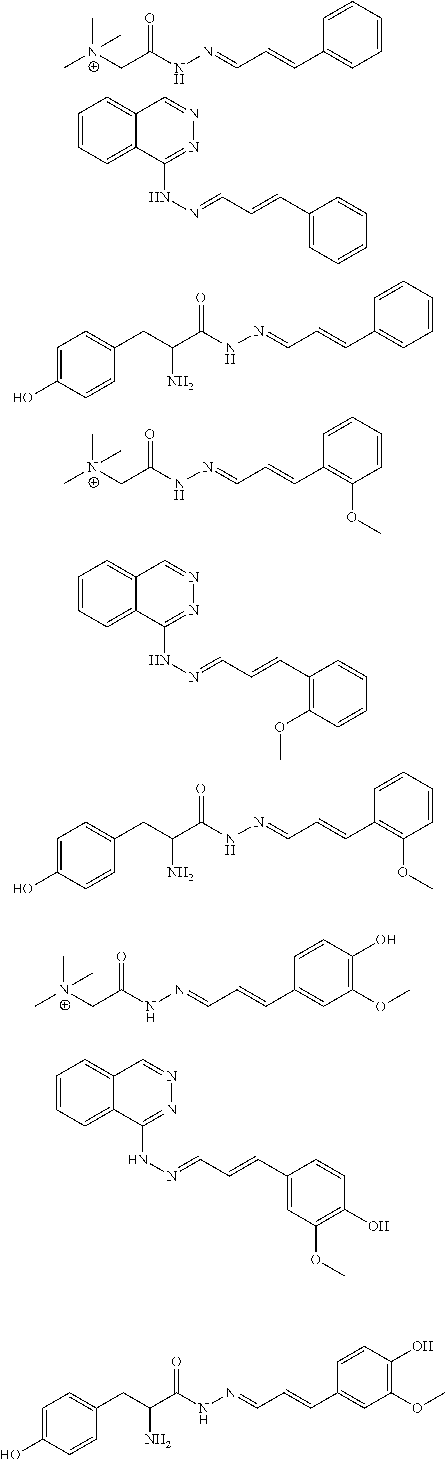

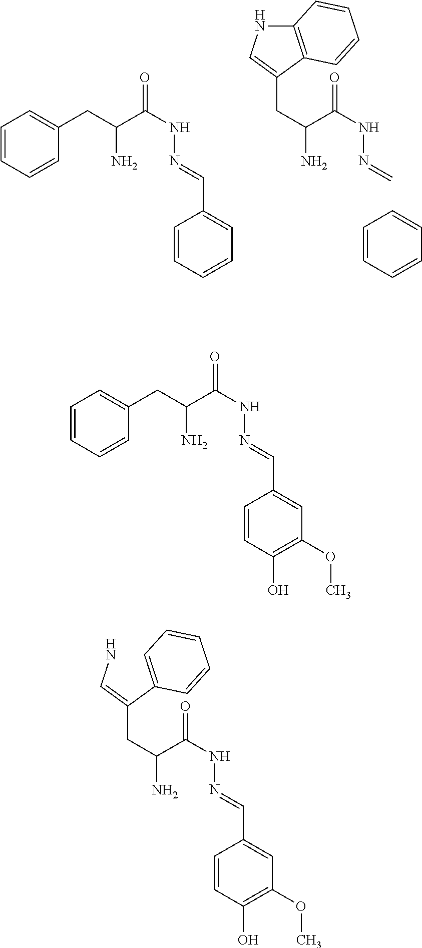

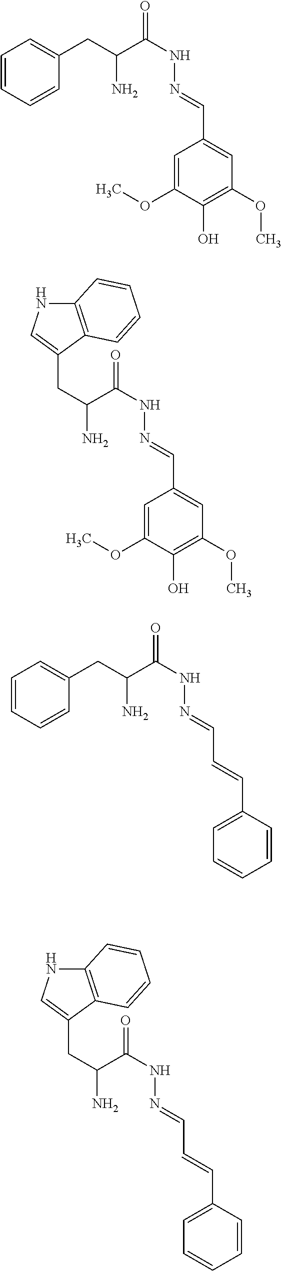

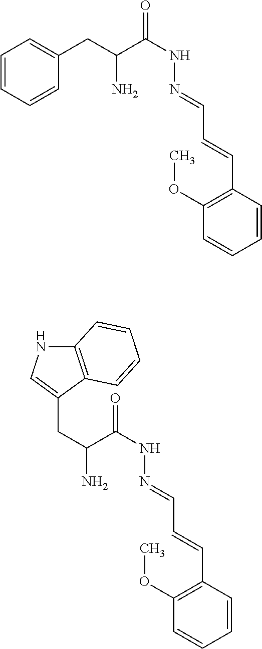 [SMILES: COc1ccccc1/C=C/C=N/NC(=O)C(N)Cc1ccccc1 ; COc1ccccc1/C=C/C=N/NC(=O)C(N)Cc1c[nH]c2ccccc12]

The above method, wherein the composition further comprises known UV absorbers.

The above method, wherein the composition further comprises known sunscreen active agents or SPF (sun protection factor) boosters.

The above method, wherein compounds of formula (I) are the sole UV absorber in the composition.

A method for providing a reversible dye to a product, said method comprising (or consisting of or consisting of) applying to said product, in an amount sufficient to provide a (possibly reversible) visible color to said product, a composition comprising (or consisting of or consisting of) a UV-inhibitor, optionally a carrier, and optionally anions; wherein said UV-inhibitor is at least one compound of formula (I)

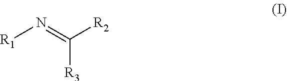

wherein R1 is an aryl derivative (is a planar aromatic compound substituted with simple R groups such as —H, alkyl groups, —OH, —OCH₃, —NH₂; for example 4-aminobenzamidine dihydrochloride)), hydralazine, pyridine, N-acyl (e.g., Girard's reagent T and Tyrosine hydrazide), guanidine or aminoguanidine group, and R2 and R3 are independently hydrogen or an aryl group substituted with a straight or branched chain alkyl (e.g., C1-10, preferably C1-6, more preferably C1-4 (e.g. —CH₃, —CH₂CH₃, —CH(CH₃)₂, —CH₂CH₂CH₃)) or an electron donating or withdrawing group (e.g., —COOH, —OH, —OCH₃, —OCH₂CH₃, —NH₂, —NHCH₃, —N(CH₃)₂, —NO₂, —Cl, —Br, —F, -Ph; specific examples include Ph-CH₃ and Ph-Cl).

The above method, wherein R1 is

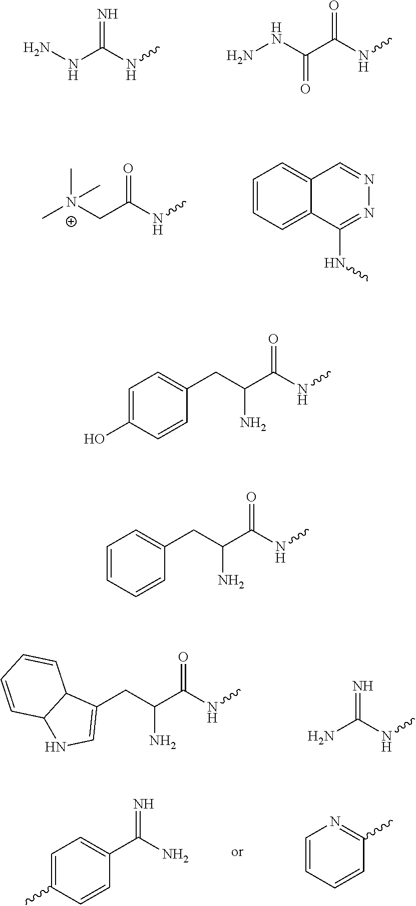

and wherein R2 and R3 are (R2 or R3 would have to be —H for any aldehyde subcomponent)

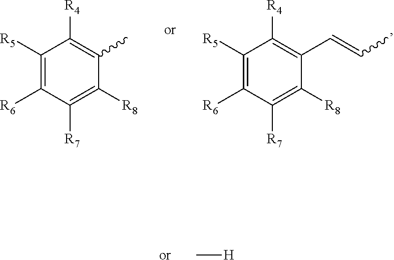

wherein R4 through R8 are independently hydrogen, a straight or branched chain alkyl (e.g., C1-10, preferably C1-6, more preferably C1-4 (e.g., —CH₃, —CH₂CH₃, —CH(CH₃)₂, —CH₂CH₂CH₃)) or an electron donating or withdrawing group (e.g., —COOH, —OH, —OCH₃, —OCH₂CH₃, —NH₂, —NHCH₃, —N(CH₃)₂, —NO₂, —Cl, —Br, —F, -Ph).

A composition comprising (or consisting of or consisting of) a UV-inhibitor, optionally a carrier, and optionally anions; wherein said UV-inhibitor is at least one compound of formula (I)

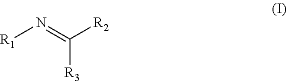

wherein R1 is an aryl derivative, N-acyl, guanidine or aminoguanidine group, and R2 and R3 are independently an aryl group substituted with a straight or branched chain alkyl (C1-10, preferably C1-6, more preferably C1-4 (e.g., —CH₃, —CH₂CH₃, —CH(CH₃)₂, —CH₂CH₂CH₃)) or an electron donating or withdrawing group (e.g., —COOH, —OH, —OCH₃, —OCH₂CH₃, —NH₂, —NHCH₃, —N(CH₃)₂, —NO₂, —Cl, —Br, —F, -Ph).

The above composition further comprising known UV absorbers.

The above composition further comprising known sunscreen active agents or SPF (sun protection factor) boosters.

The above composition, wherein compounds of formula (I) are the sole UV absorber in the composition.

The above composition, wherein compounds of formula (I) are made by the general scheme in FIG. 1A or FIG. 1B.

The above composition, wherein compounds of formula (I) do not include compounds known before the filing date of this provisional application.

The term “consisting essentially of” excludes additional method (or process) steps or composition components that substantially interfere with the intended activity of the method (or process) or composition, and can be readily determined by those skilled in the art (for example, from a consideration of this specification or practice of the invention disclosed herein).

The invention illustratively disclosed herein suitably may be practiced in the absence of any element (e.g., method (or process) steps or composition components) which is not specifically disclosed herein. Thus, the specification includes disclosure by silence (“Negative Limitations In Patent Claims,” AIPLA Quarterly Journal, Tom Brody, 41(1): 46-47 (2013): “ . . . Written support for a negative limitation may also be argued through the absence of the excluded element in the specification, known as disclosure by silence . . . Silence in the specification may be used to establish written description support for a negative limitation. As an example, in Ex parte Fin [No. 2009-0486, at 2, 6 (B.P.A.I. May 7, 2009)] the negative limitation was added by amendment . . . In other words, the inventor argued an example that passively complied with the requirements of the negative limitation . . . was sufficient to provide support . . . This case shows that written description support for a negative limitation can be found by one or more disclosures of an embodiment that obeys what is required by the negative limitation . . . . ”

Other embodiments of the invention will be apparent to those skilled in the art from a consideration of this specification or practice of the invention disclosed herein. It is intended that the specification and examples be considered as exemplary only, with the true scope and spirit of the invention being indicated by the following claims.

TABLE 1
Skin permeation coefficients (Kₚ) of aminoguanidine
and aldehyde subcomponents, structurally similar
guanyl hydrazones, and commercial UV-absorbers.

| | | | Kₚ | Kₚ/ |
|---|---|---|---|---|
| Compound | K_{ow} | MW (g) | (cm/hr) | K_{p octinoxate} |
| Subcomponents | | | | |
| Aminoguanidine | −1.76 | 74.09 | 0.00005 | 0.0003 |
| Diaminoguanidine | −2.19 | 89.10 | 0.00002 | 0.0001 |
| Cinnamaldehyde | 2.91 | 148.21 | 0.01873 | 0.1276 |
| Cuminaldehyde | 1.47 | 106.12 | 0.00418 | 0.0285 |
| Benzaldehyde | 1.85 | 132.16 | 0.00463 | 0.0316 |
| Anisaldehyde | 1.64 | 136.15 | 0.00309 | 0.0211 |
| Structurally Similar | | | | |
| Robenidine | 1.66 | 231.08 | 0.00073 | 0.0050 |
| Guanabenz | 3.68 | 334.20 | 0.00498 | 0.0339 |
| Commercial | | | | |
| Octinoxate | 5.49 | 290.40 | 0.14678 | 1.0000 |

TABLE 2
Toxicity and biodegradation of some possible subcomponents,
color-coded according to U.S. EPA hazard categorization.

| | LD50 | LC50 | Biodegradation |
|---|---|---|---|
| | (mg/kg) | (mg/L) | half- |
| Compound | Rat, oral | Fish | life (days) |
| Aminoguanidine | 5000 | 1585 mg/L | 4.52 |
| (bicarbonate) | | Zebra fish | |
| Diaminoguanidine | No Data | No Data | 6.66 |
| Cinnamaldehyde | 3350 | 105.8 mg/L | 4.82 |
| | | Fathead minnow | |
| Cuminaldehyde | 1390 | 6.62 mg/L | 3.72 |
| | | Fathead minnow | |
| Benzaldehyde | 1300 | 12.4 mg/L | 5.60 |
| | | Fathead minnow | |
| Anisaldehyde | 1510 | 215 mg/L | 4.31 |
| | | Silver ide | |

We claim:

1. A method for protecting a product from UV light, said method comprising applying to said product, in an amount sufficient to protect said product from UV light, a composition comprising a UV-inhibitor, optionally a carrier, and optionally anions; wherein said UV-inhibitor is at least one compound of formula (I) 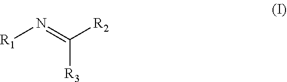
wherein R1 is an aryl derivative, hydralazine, pyridine, N-acyl, guanidine or aminoguanidine group, and R2 and R3 are independently hydrogen or an aryl group substituted with a straight or branched chain alkyl or an electron donating or withdrawing group.

2. The method according to claim 1, wherein R1 is 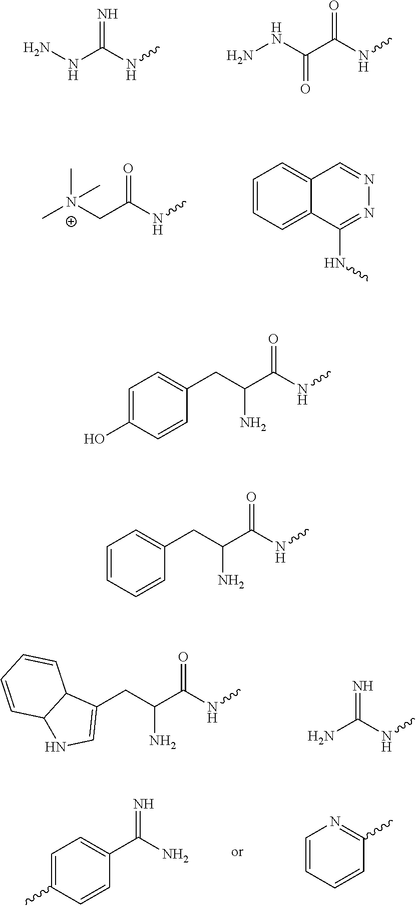
and wherein R2 and R3 are 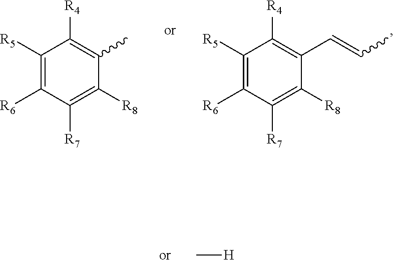
wherein R4 through R8 are independently hydrogen, a straight or branched chain alkyl, or an electron donating or withdrawing group.

3. A composition comprising a UV-inhibitor, optionally a carrier, and optionally anions;
wherein said UV-inhibitor is at least one compound of formula (I) 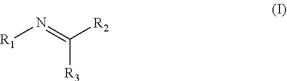
wherein R1 is an aryl derivative, N-acyl, guanidine or aminoguanidine group, and R2 and R3 are independently an aryl group substituted with a straight or branched chain alkyl or an electron donating or withdrawing group.

4. A method for providing a reversible dye to a product, said method comprising applying to said product, in an amount sufficient to provide a visible color to said product, a composition comprising a UV-inhibitor, optionally a carrier, and optionally anions; wherein said UV-inhibitor is at least one compound of formula (I) 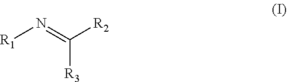
wherein R1 is an aryl derivative, amino aryl, hydralazine, pyridine, N-acyl, guanidine or aminoguanidine group, and R2 and R3 are independently hydrogen, an aryl group substituted with a straight or branched chain alkyl or an electron donating or withdrawing group.
